(19) United States
(12) Patent Application Publication
(10) Pub. No.: US 2018/0098950 A1
(43) Pub. Date: Apr. 12, 2018

(54) INHIBITORS OF ABETA AND SYNUCLEIN AGGREGATION

(71) Applicant: The Feinstein Institute For Medical Research, Manhasset, NY (US)

(72) Inventor: Yousef Al-Abed, Locust Valley, NY (US)

(73) Assignee: The Feinstein Institute For Medical Research, Manhasset, NY (US)

(21) Appl. No.: 15/838,780

(22) Filed: Dec. 12, 2017

Related U.S. Application Data
(63) Continuation of application No. 12/308,475, filed on Feb. 10, 2009.
(60) Provisional application No. 60/816,132, filed on Jun. 23, 2006.

(51) Int. Cl. A61K 31/165

(52) U.S. Cl. CPC A61K 31/165

(57) ABSTRACT

Provided are methods of inhibiting aggregation of amyloid-beta (Aβ) or accumulation of aggregated Aβ using certain guanylhydrazone compounds. Also provided are methods of treating or preventing an amyloid-related disease in a mammal, methods of treating a subject having Alzheimer's disease, methods of treating a subject at risk for Alzheimer's disease, methods of inhibiting aggregation or accumulation of a synuclein, methods of treating a subject having a disease at least partially mediated by synuclein, methods of treating a subject at risk for a disease at least partially mediated by synuclein, and methods of inhibiting aggregation or accumulation of a protein involved in a conformational disease, using the guanylhydrazone compounds.

### CROSS-REFERENCE TO RELATED APPLICATION

This application claims the benefit of U.S. Provisional Application No. 60/816,132, filed Jun. 23, 2006.

### BACKGROUND OF THE INVENTION

### (1) Field of the Invention

The present invention generally relates to treatments for diseases involving aggregating proteins. More specifically, the invention is directed to methods of inhibiting aggregation of those proteins, and the accumulation of such protein aggregates, using certain compounds.

### (2) Description of the Related Art

Over the past 20 years, a great deal of research has investigated the effects of the approximately 4-kDa amyloid β protein (Aβ) in the development of Alzheimer's disease (AD). In the brains of patients with AD, Aβ accumulates as amyloid in senile plaques and in the walls of cerebral blood vessels as well as in more diffuse immunoreactive deposits. This accumulation is thought to result in a pathological cascade that ultimately results in neuronal dysfunction and cell death (Selkoe, 2001; Hardy and Higgins, 1992). Multiple Aβ species with various amino and carboxyl termini are generated from the amyloid 3 protein precursor (APP) through sequential proteolytic cleavages by the β- and γ-secretases (Golde et al., 2000). The 40-amino acid form (Aβ340) is the most abundantly produced Aβ peptide, whereas a slightly longer and less abundant 42-amino acid form (Aβ42) has been implicated as the more pathogenic species (Younkin, 1998). Under in vitro conditions, Aβ42 forms aggregates much more readily than Aβ40 and other shorter Aβ peptides, and these aggregates are toxic to a variety of cells in culture. Despite being a minor Aβ species, Aβ42 is deposited earlier and more consistently than Aβ40 in the AD brain.

In addition to AD deposition, neurofibrillary tangle accumulation, and neuronal loss, the end-stage pathology of AD is also notable for the presence of numerous cellular and molecular markers of an inflammatory response that are often associated with the AD deposits (Akiyama et al., 2000). The cellular inflammatory response consists of widespread astrogliosis and microgliosis. A large number of molecular markers of inflammation are also increased, including multiple cytokines, interleukins, other acute-phase proteins, and complement components. Aβ aggregates appear capable of inciting an inflammatory response, and there is evidence that inflammation can promote increased Aβ production and also enhance Aβ deposition (Id.). Thus, an Aβ-induced inflammatory response could promote further Aβ accumulation and increased inflammation. Alternatively, it is possible that under certain circumstances the inflammatory response is beneficial and may actually promote Aβ clearance (Wyss-Coray et al., 2002).

In light of the notion that the inflammatory response to Aβ is detrimental, anti-inflammatory drugs have been suggested as beneficial agents in AD therapy (Aisen, 1997; McGeer et al., 1996). This idea is supported by epidemiologic data, which consistently show that long-term use of nonaspirin NSAIDs is associated with protection from the development of AD (Mc Geer et al., 1996; in t'Veld et al., 2001; Stewart et al., 1997; Zandi et al., 2002). Indeed, this evidence has been used as the rationale for previous and ongoing trials of select NSAIDs in AD. CNI-1493 is a tetravalent guanylhydrazone that inhibits phosphorylation of p38 MAPK, c-Raf, and suppresses proinflammatory cytokine release from monocytes and macrophages (Cohen et al., 1997; Lowenberg et al., 2005; Bianchi et al., 1996; Wang et al., 1988; Tracy, 1998). Systemic administration of CNI-1493 is effective in the treatment of experimental autoimmune encephalomyelitis, cerebral ischemia, Crohn's disease, and arthritis (Martiney et al., 1998; Meistrell et al., 1997; Lowenberg et al., 2005; Akerlund et al., 1999).

### SUMMARY OF THE INVENTION

Accordingly, the inventor has discovered that certain compounds inhibit aggregation of various proteins, such as amyloid-beta (Aβ) and synuclein, and the accumulation of such protein aggregates. Thus, the invention is directed to methods of inhibiting aggregation of amyloid-beta (Aβ) or accumulation of aggregated Aβ. The methods comprise contacting the Aβ with Compound I in a manner sufficient to inhibit aggregation of Aβ or accumulation of aggregated Aβ. In these methods, Compound I is

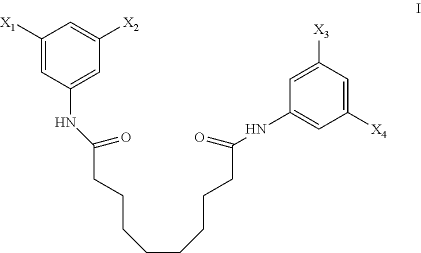

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H.

The invention is also directed to methods of treating or preventing an amyloid-related disease in a mammal. The methods comprise administering Compound I to the mammal in a manner sufficient to treat or prevent the disease, where Compound I is

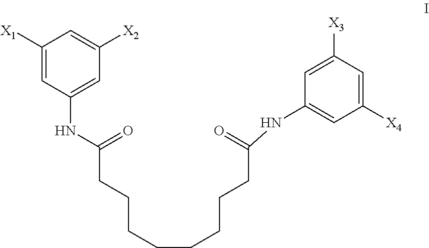

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H.

The invention is additionally directed to methods of treating a subject having Alzheimer's disease. The methods comprise administering Compound I to the subject in a manner sufficient to treat the disease, where Compound I is

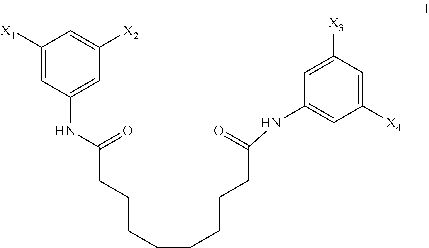

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

Additionally, the invention is directed to methods of treating a subject at risk for Alzheimer's disease. The methods comprise administering Compound I to the subject, where Compound I is

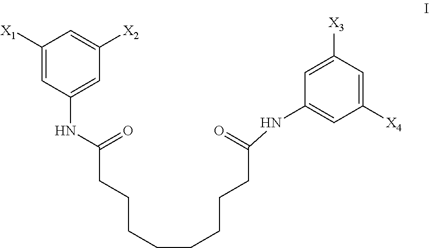

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N(CH₃)—, redGhyCH is NH₂(CNH)—NH—NH—CH₂— and red GhyCCH₃ is NH₂(CNH)—NH—NH—CH(CH₃)— provided at least one of X₁, X₂, X₃ and X₄ is not H.

The invention is further directed to methods of inhibiting aggregation or a synuclein and/or accumulation of an aggregated synuclein. The methods comprise contacting the synuclein with Compound I in a manner sufficient to inhibit aggregation or accumulation of the synuclein, wherein Compound I is

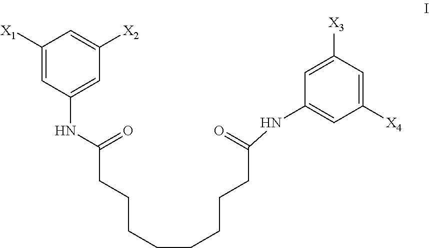

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

The invention is also directed to methods of treating a subject having a disease at least partially mediated by synuclein. The methods comprise administering Compound I to the subject, where Compound I is

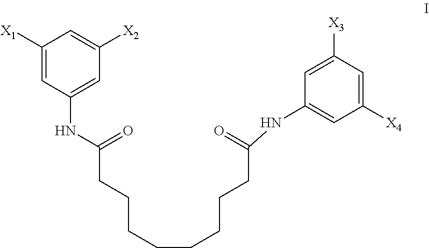

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

Additionally, the invention is directed to methods of treating a subject at risk for a disease at least partially mediated by synuclein. The methods comprise administering Compound I to the subject, where Compound I is

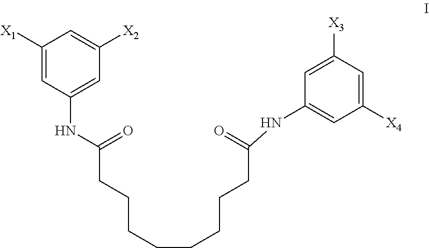

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

The invention is further directed to methods of inhibiting aggregation of a protein and/or accumulation of aggregates of a protein involved in a conformational disease. The methods comprise contacting the protein with Compound I in a manner sufficient to inhibit aggregation or accumulation of the protein, where Compound I is

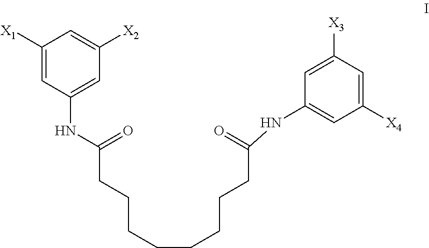

wherein

X₁, X₂, X₃ and X₄ is independently GhyCH—, GhyCCH₃—, redGhyCH— or redGhyCCh₃— or H, where GhyCH is NH₂(CNH)—NH—NH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

The invention is additionally directed to the use of Compound I for the manufacture of a medicament for the treatment or prevention of an amyloid—related disease in a mammal. Here, Compound I is

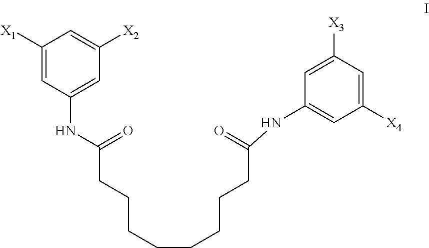

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

The invention is further directed to the use of Compound I for the manufacture of a medicament for treating a subject having Alzheimer's disease. For these uses, Compound I is

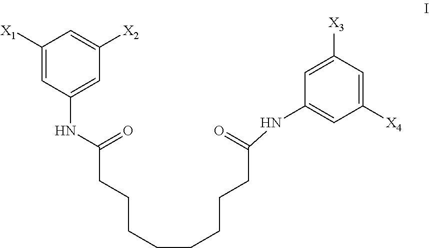

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

Additionally, the invention is directed to the use of Compound I for the manufacture of a medicament for treating a subject having a disease at least partially mediated by a synuclein. Here, Compound I is

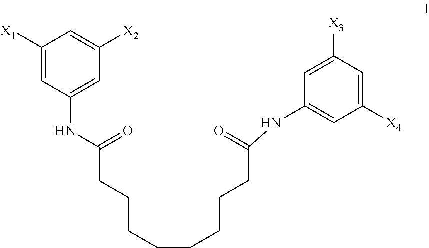

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

Further, the present invention is directed to the use of compound I for treating or preventing an amyloid-related disease in a mammal. Here, Compound I is

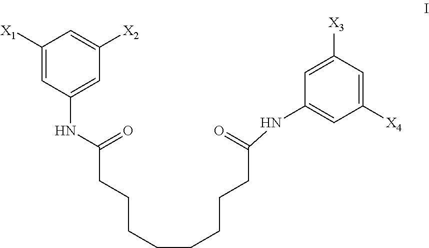

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H.

Also, the invention is directed to the use of Compound I for treating a subject having Alzheimer's disease. Here, Compound I is

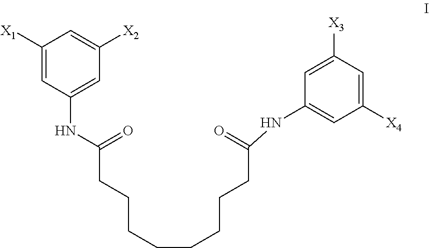

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)——NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H.

The present invention is additionally directed to the use of Compound I for treating a subject having a disease at least partially mediated by synuctein. For these uses, Compound I is

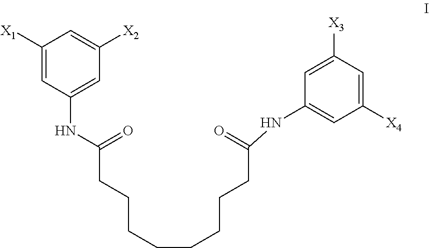

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H.

The invention is further directed to the use of Compound I for the manufacture of a medicament for treating a subject having a conformational disease. Here, Compound I is

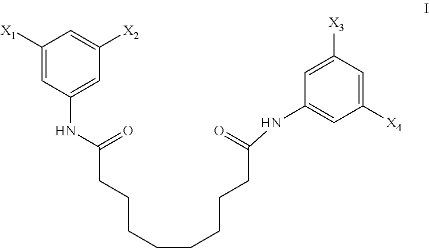

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

### BRIEF DESCRIPTION OF THE DRAWINGS

FIG. 1 is micrographs of experimental results showing that CNI-1493 reduces Aβ plaque pathology in transgenic APP-expressing TgCRND8 mice. The TgCRND8 mice were treated with CNI-1493 or vehicle, then killed after 2 months of treatment. Sagittal sections from vehicle- or CNI-1493-treated mice were immunohistochemically stained for Aβ using the mouse anti-human Aβ monoclonal antibody 6F/3D.

FIG. 2 is graphs of experimental results showing Aft plaque evaluation in vehicle and CNI-1493 treated transgenic APP-expressing TgCRND8 mice shows a profound reduction of plaque deposition after CNI-1493 treatment. Digital images from cortex and hippocampus were obtained and analyzed with image analysis software “SIS analysis Auto Software 3.2”. Plaque number was calculated by the number of plaques divided by the area of interest in square millimeters. CNI-1493 reduced the plaque number in the cortex by 57% (p<0.01), and in the hippocampus by 60% (p<0.01). The plaque area was computed and expressed as plaque area in square micrometers per area of interest in square millimeters. CNI-1493 reduced plaque area in cortex by 70% (p<0.01) and in hippocampus by 86% (p<0.01). Each column represents 4 animals.

FIG. 3 is western blots showing the effect of CNI-1493 on soluble Aβ in transgenic APP-expressing TgCRND8 mice. Panel A. 20 μg protein per lane was separated by pre-cast NuPAGE Novex 4-12% Bis-Tris gels and transferred onto nitrocellulose membranes using the XCell II™ blot system. For the detection of membrane-bound soluble Aβ, 6E10 monoclonal antibodies were used. A massive loss of soluble Aβ isoforms was measured in the brains of two of the four CNI-1493-treated animals. Equal protein loading was assessed by reprobing the membrane with monoclonal antibodies against GAPDH (Panel B).

FIG. 4 is a western blot showing that CNI-1493 deactivates microglial cells in CNI-1493 treated transgenic APP-expressing TgCRND8 mice. 60 μg protein per lane was separated by pre-cast NuPAGE Novex 4-12% Bis-Tris gels and transferred onto nitrocellulose membranes using the XCell II™ blot system. The activation of glial cells was assessed by staining for the macrophage activation with antibodies against the F4/80 antigen. Western blot analysis revealed a decline of F4/80 in all CNI-1493 animals. Equal protein loading was assessed by reprobing the membrane with monoclonal antibodies against GAPDH.

FIG. 5 is western blots showing the effect of CNI-1493 on APP processing in N2a cells expressing wild type APP695. Cells were treated for 24 h with the indicated concentrations of CNI-1493. Medium was changed and drug treatment was continued for another 4 h to allow Aβ secretion. Total secreted Aβ (total sAβ) was analyzed by western blot using 6E10 antibody (panel a). APP C-terminal fragments, C99 (panel b) and C83 (panel c) were analyzed using 6E10 and R1 antibodies, respectively. Full length APP (panel d) was tested with antibodies LN27.

FIG. 6 is a graph of experimental results showing that CNI-1493 prevents aggregation of Aβ42, Aβ40 and synuclein, as shown by CNI-1492 preventing recognition by an anti-oligomer antibody.

FIG. 7 is graphs and electron micrographs of experimental results, showing that exposure of Aβ to CNI-1493 disrupts Aβ oligomer assembly. Panel A is a graph showing the reduction in recognition of Aβ oligomers in an ELISA using anti-Aβ oligomer antibody (shown as reduced optical density [OD] in the ELISA) in a solution with increasing CNI-1493 concentrations. Panel B is electron micrographs showing inhibited Aβ42 oligomer fibrilization with exposure to CNI-1493. Panel C is a graph showing an increase in cell viability of Aβ-exposed neuroblastoma cells on addition of increasing concentrations of CNI-1493.

### DETAILED DESCRIPTION OF THE INVENTION

Accordingly, the inventor has discovered that certain compounds inhibit aggregation of various proteins, such as amyloid beta (Aβ) and synuclein, both in vitro and in vivo. See Examples. In those Examples, this inhibition of aggregation was demonstrated using the compound CNI-1493, or N,N′-bis(3,5-diacetylphenyl)decanediamide tetrakis(amidinohydrazone) tetrahydrochloride. Additionally, a broader class of guanylhydrazone compounds that includes CNI-1493 is known to have properties similar to CNI-1493. See U.S. Pat. No. 5,599,984, 5,750,573, 5,753,684, 5,849,794, 5,854,289, 5,859,062, 6,008,255, 6,022,900, 6,180,676 B1 and 6,248,787 B1.

Thus, the invention is directed to methods of inhibiting aggregation of amyloid beta (Aβ) or accumulation of aggregated Aβ. The methods comprise contacting the Aβ with Compound I in a manner sufficient to inhibit aggregation of Aβ or accumulation of aggregated Aβ. In these methods, Compound I is

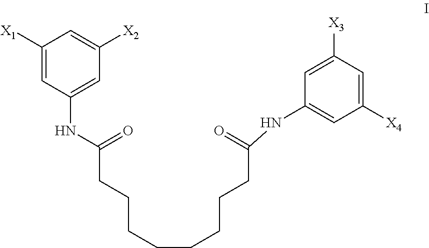

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H. Preferably the Compound I used in these methods is N,N′-bis(3,5-diacetylphenyl)decanediamide tetrakis(amidinohydrazone) tetrahydrochloride.

These methods can be used on Aβ that is outside of a living mammal, or, preferably on Aβ that is part of a living mammal. The methods are useful for any form of Aβ, including Aβ40 and Aβ42. Where the method is used on a living mammal, the mammal is preferably at risk for Alzheimer's disease or has Alzheimer's disease. Most preferably, the mammal is a human.

As used herein, “Alzheimer's disease” is the familiar human disease characterized by neurofibrillary plaques made of Aβ peptides, as well as any of the known animal models of that disease (see, e.g., Example 1).

When used on a living mammal, the compound in these methods are preferably formulated in a pharmaceutically acceptable excipient. By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The above-described compounds can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the compound. As used herein, nasally administering or nasal administration includes administering the compound to the mucous membranes of the nasal passage or nasal cavity of the patient. As used herein, pharmaceutical compositions for nasal administration of the compound include therapeutically effective amounts of the compound prepared by well known methods to be administered, for example, as a nasal spray, nasal drop, suspension, gel, ointment, cream or powder. Administration of the compound may also take place using a nasal tampon or nasal sponge.

Where the compound is administered peripherally such that it must cross the blood-brain barrier, the compound is preferably formulated in a pharmaceutical composition that enhances the ability of the compound to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The compound can be combined with one or several types of micelles, and can further be contained within the micelles or associated with their surface.

Alternatively, the compound can be combined with liposomes (lipid vesicles) to enhance absorption. The compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

The invention is also directed to methods of treating or preventing an amyloid-related disease in a mammal. The methods comprise administering Compound I to the mammal in a manner sufficient to treat or prevent the disease, where Compound I is

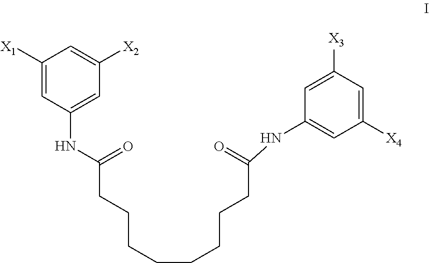

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H. Preferably, Compound I is N,N′-bis(3,5-diacetylphenyl)decanediaminde tetrakis(amidinohydrazone) tetrahydrochloride.

In these methods, the mammal preferably is at risk for Alzheimer's disease, or has Alzheimer's disease. The mammal is most preferably a human.

The invention is additionally directed to methods of treating a subject having Alzheimer's disease. The methods comprise administering Compound I to the subject in a manner sufficient to treat the disease, where Compound I is

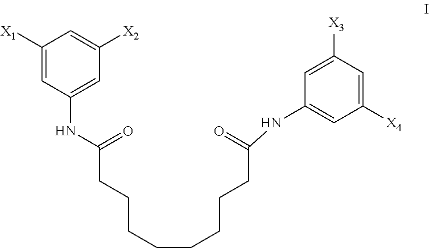

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. As in the methods described above, Compound I is preferably N,N′-bis(3,5-diacetylphenyl)decanediamide tetrakis(amidinohydrazone) tetrahydrochloride.

Additionally, the invention is directed to methods of treating a subject at risk for Alzheimer's disease. The methods comprise administering Compound I to the subject, where Compound I is

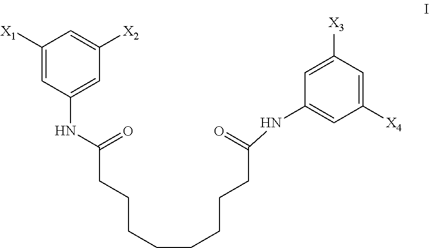

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. As in the methods described above, Compound I is preferably N,N′-bis(3,5-diacetylphenyl)decanediamide tetrakis(amidinohydrazone) tetrahydrochloride.

The invention is further directed to methods of inhibiting aggregation or a synuclein and/or accumulation of an aggregated synuclein. The methods comprise contacting the synuclein with Compound I in a manner sufficient to inhibit aggregation or accumulation of the synuclein, wherein Compound I is

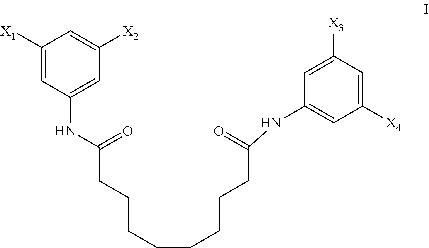

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. Compound I in these methods is preferably N,N′-bis(3,5-diacetylphenyl)decanediamide tetrakis(amidinohydrazone) tetrahydrochloride.

These methods can be used on synuclein that is outside of a living mammal, or, preferably on synuclein that is part of a living mammal. Where the method is used on a living mammal, the mammal preferably has or is at risk for a disease at least partially mediated by synuclein. Examples of such diseases are Parkinson's disease and certain neurodegenerative diseases. Thus, more preferably, the mammal has or is at risk for Parkinson's disease or a neurodegenerative disease. Most preferably, the mammal has or is at risk for Parkinson's disease.

The invention is also directed to methods of treating a subject having a disease at least partially mediated by synuclein. The methods comprise administering Compound I to the subject, where Compound I is

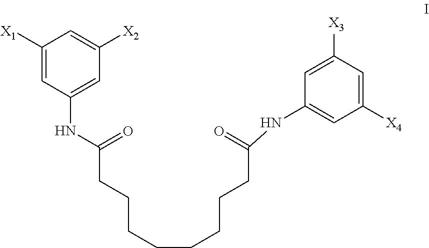

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. Preferably, Compound I is N,N′-bis(3,5-diacetylphenyl)decanediamide tetrakis(amidinohydrazone) tetrahydrochloride. More preferably, the disease at least partially mediated by synuclein is Parkinson's disease or a neurodegenerative disease, most preferably Parkinson's disease.

Additionally, the invention is directed to methods of treating a subject at risk for a disease at least partially mediated by synuclein. The methods comprise administering Compound I to the subject, where Compound I is

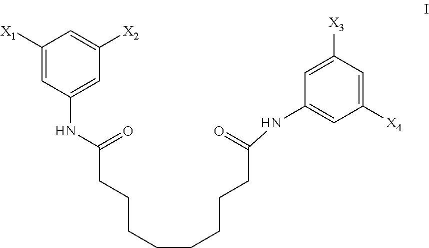

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H.

Since the guanylhydrazone compounds in the above-described methods inhibit aggregation of Aβ as well as synuclein, the compounds appear to generally inhibit aggregation or accumulation of proteins involved in conformational disease. As used herein, “conformational disease” is a disease involving at least one misfolded protein or peptide. Examples of proteins involved in conformational diseases include serpin, prions, glutamine repeat proteins, tau proteins, hemoglobin, synuclein, immunoglobulin light chains, serum amyloid A proteins, a β₂ microglobulin, cystatin C, huntingtin, apolipoprotein A1, lysozymes, transthyretins, Aβs, β-amyloid peptide, procalcitonin, amylin, and islet amyloid polypeptide. Examples of conformational diseases include Parkinson's disease, Alzheimer's disease, prion diseases, and type 2 diabetes mellitus.

Thus, the invention is further directed to methods of inhibiting aggregation or accumulation of a protein involved in a conformational disease. The methods comprise contacting the protein with Compound I in a manner sufficient to inhibit aggregation or accumulation of the protein, where Compound I is

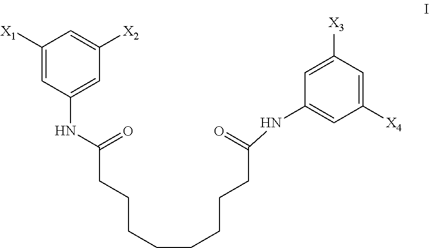

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. Preferably, Compound I is N,N′-bis(3,5—diacetylphenyl)decanediamide tetrakis(amidinohydrazone) tetrahydrochloride.

The protein in these methods is preferably a serpin, a prion, a glutamine repeat protein, a tau hemoglobin, a synuclein, an immunoglobulin light chain, a serum amyloid A protein, a β₂ microglobulin, a cystatin C, a huntingtin, a apolipoprotein Al, a lysozyme, a transthyretin, an Aβ, a β-amyloid peptide, a procalcitonin, an amylin or an islet amyloid polypeptide.

These methods can be used on proteins that are outside of a living mammal, or, preferably on proteins that is part of a living mammal. Where the method is used on a living mammal, the mammal preferably has or is at risk for a disease at least partially mediated by the protein. Most preferably, the mammal has or is at risk for Parkinson's disease, Alzheimer's disease, a prion disease, or type 2 diabetes mellitus.

The invention is additionally directed to the use of Compound I for the manufacture of a medicament for the treatment or prevention of an amyloid-related disease in a mammal. Here, Compound I is

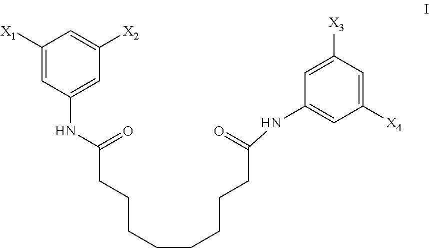

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X2, X3 and X4 is not H.

For these uses, Compound I is preferably N,N′-bis(3,5-diacetylphenyl)decanediamide tetrakis(amidinohydrazone) tetrahydrochloride. It is also preferred that the mammal is at risk for Alzheimer's disease or has Alzheimer's disease.

These uses can be applied to any mammal. Preferably, the mammal is a human.

The invention is further directed to the use of Compound I for the manufacture of a medicament for treating a subject having Alzheimer's disease. For these uses, Compound I is

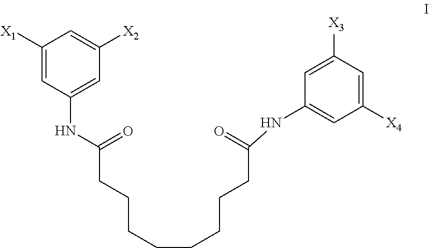

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. Preferably, Compound I is N,N′-bis(3,5-diacetylphenyl)decanediamide tetrakis(amidinohydrazone) tetrahydrochloride. It is also preferred that the mammal is a human.

Additionally, the invention is directed to the use of Compound I for the manufacture of a medicament for treating a subject having a disease at least partially mediated by a synuclein. Here, Compound I is

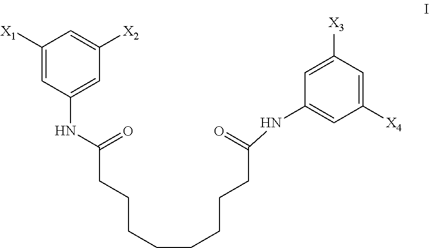

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. Preferably here, Compound I is N,N′-bis(3,5-diacetylphenyl)decanediamide tetrakis(amidinohydrazone) tetrahydrochloride. It is also preferred that the disease is Parkinson's disease or a neurodegenerative disease. Additionally, the disease is preferably at least partially mediated by synuclein is Parkinson's disease.

Further, the present invention is directed to the use of compound I for treating or preventing an amyloid-related disease in a mammal. Here, compound I is

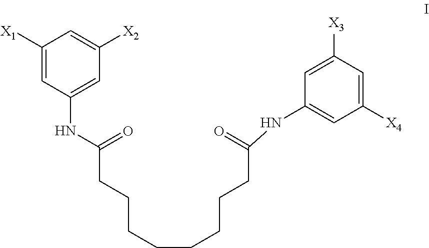

wherein

X₁, X2, X3 and X₄ is independently GhyCH—, GhyCCH₃—, redGhyCH— or redGhyCCh₃— or H, where GhyCH is NH₂(CNH)—NH—N—CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X4 is not H. Preferably, Compound I is N,N′-bis(3,5-diacetylphenyl)decanediamide tetrakis(amidinohydrazone) tetrahydrochloride.

Also, the invention is directed to the use of Compound I for treating a subject having Alzheimer's disease. Here, Compound I is

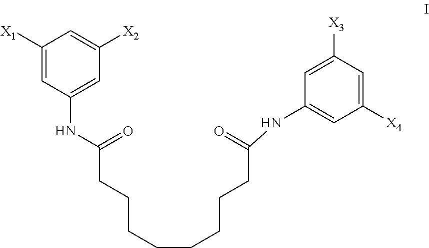

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X₄ is not H. Preferably, Compound I is N,N′-bis(3,5-diacetylphenyl)decanediamide tetrakis(amidinohydrazone) tetrahydrochloride.

The present invention is additionally directed to the use of Compound I for treating a subject having a disease at least partially mediated by synuclein. For these uses, Compound I is

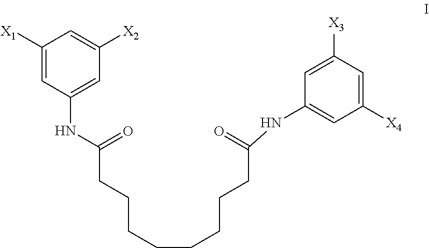

wherein

X₁, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)—, provided at least one of X₁, X₂, X₃ and X4 is not H. Preferably, Compound I is N,N′-bis(3,5-diacetylphenyl)decanediamide tetrakis(amidinohydrazone) tetrahydrochloride.

The invention is further directed to the use of Compound I for the manufacture of a medicament for treating a subject having a conformational disease. Here, Compound I is

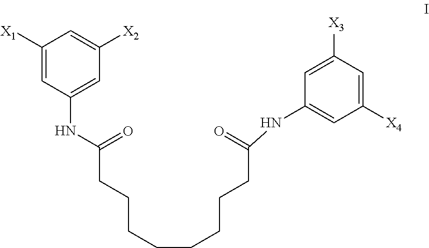

wherein

X1, X₂, X₃ and X₄ is independently GhyCH—, GhyCCH₃—, redGhyCH— or redGhyCCh₃— or H, where GhyCH is NH₂(CNH)—NH—N═CH—. GhyCCH₃ is NH₂(CNH)—NH—N═C(CH₃)—, redGhyCH is NH₂(CNH)—NH—NH—CH₂— and redGhyCCH₃ is NH₂(CNH)—NH—NH—CH(CH₃)— provided at least one of X₁, X₂, X₃ and X₄ is not H. Preferably, Compound I is N,N′-bis(3,5-diacetylphenyl)decanediamide tetrakis(amidinohydrazone) tetrahydrochloride. It is also preferred that the conformational disease involves aggregation of a protein, where the protein is preferably a serpin, a prion, a glutamine repeat protein, a tau hemoglobin, a synuclein, an immunoglobulin light chain, a serum amyloid A protein, a β₂ microglobulin, a cystatin C, a huntingtin, a apolipoprotein A1, a lysozyme, a transthyretin, an Aβ, a β-amyloid peptide, a procalcitonin, an amylin or an islet amyloid polypeptide.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1.

### CNI-1493 Inhibits Aβ Production and Prevents Plaque Formation in an Animal Model of Alzheimer's Disease

### Example Summary

Alzheimer's disease (AD) is characterized by a microglial-mediated inflammatory response elicited by extensive amyloid deposition in the brain. Nonsteroidal anti-inflammatory drug treatment reduces AD risk, slows disease progression, and reduces microglial activation; however, the molecular basis of these effects is unknown. We report that treatment of 4-month-old TgCRND8 mice overexpressing human amyloid precursor protein (APP) with the potent macrophage deactivation agent CNI-1493 for an treatment period of only 8 weeks resulted in the dramatic reduction of Aβ deposition. CNI-1493 treatment resulted in 70% reduction of amyloid plaque area in the cortex and 87% reduction in the hippocampus of these animals. In addition, CNI-1493 treatment resulted in a significant reduction in microglial activation in the TgCRND8 mice, as measured by F4/80 expression.

Our in vitro analysis of CNI-1493 treatment on APP processing in an APP overexpressing cell line suggests a profound dose-dependent decrease of total Aβ accumulation. This effect appears to be completely unrelated from both the production of APP and changed β- or γ-secretase activities.

This study identifies the anti-inflammatory agent CNI-1493 as a very promising candidate for the treatment and prevention of AD.

### Introduction

The aim of this study was to test whether CNI-1493 acted to suppress the development of amyloid pathology and inflammatory responses in the brains of APP-expressing TgCRND8 transgenic mice. The studies described here establish that only two months of CNI-1493 treatment resulted in a massive reduction in the plaque burden in these mice and a reduction in microglial activation.

### Materials and Methods

Animals. All animal procedures were approved by the office of the district president and the institutional animal care and use committee for the University of Munster. We used the TgCRND8 mouse line (courtesy of David Westaway, University of Toronto, Toronto, Ontario, Canada). TgCRND8 mice encode a double mutant form of amyloid precursor protein 695 (KM670/671 NL+V717F) under the control of the PrP gene promoter (Chishti et al., 2001). Thioflavine S-positive Aβ amyloid deposits are present at 3 months, with dense-cored plaques and neuritic pathology evident from 5 months of age. TgCRND8 mice exhibit 3,200-4,600 pmol of Aβ per g brain at age 6 months, with an excess of Aβ42 over Aβ40.

Drug treatment of TgCRND8 mice. APP transgenic TgCRND8 mice at 4 months old received twice a week an i.p. injection of 200 μl containing 200 μg CNI-1493 (8 mg/kg) for 8 weeks. There were four animals in each treatment group. Animals were housed singly in individual cages. At the end of the experimental period, animals were killed by decapitation. The brain was dissected and the hemispheres separated along the midline. One hemisphere was fixed in 4% buffered formaldehyde for 24 h followed by dehydration and paraffin embedding. The other hemisphere was immediately snap-frozen in liquid nitrogen and kept at −80° C.

Immunohistochemistry. Three pairs of 2 μm sagittal brain sections of each transgenic animal were stained for Aβ immunoreactivity. The pairs (10 μm distance) were situated 100 μm, 200 μm and 300 μm lateral from the mid-sagittal fissure. All slices were pretreated with formic acid and automatically stained in a TechMate Instrument (Dako Cytomation, Hamburg, Germany) with 6F/3D anti-Aβ monoclonal antibody to residues 8-17 (Dako, 1:100). For further steps, the Dako StreptABC complex-horseradish peroxidase conjugated “Duet” anti mouse/rabbit antibody kit was used and developed with 3, 3′-diaminobenzidine (DAB) as chromogen. Counterstaining was performed with hematoxylin. All slides were stained in two consecutive procedures making sure that brains of both experimental groups were equally distributed in both procedures.

Image analysis. To quantify Aβ plaque burden, cortices and hippocampi of all stained sections were digitalized by a ColorView II, 3, 3 Mega Pixel CCD camera under constant light and filter settings. Color images were converted to greyscale to obtain best contrast between positive immunoreactivity and background. A constant threshold was chosen for all images to detect immunoreactive staining.

Morphometric measurements were performed by image analysis software “SIS analysis Auto Software 3.2” (Soft Imaging System GmbH, http://www.soft-imaging.org/). Total number and surface of plaques was related to the total area analyzed.

Biochemical analysis. Half of the brain was weighed and homogenized in an appropriate volume of T-PER (Perbio, Bonn, Germany) in accordance to the guidelines of the manufacturer. Lysate protein concentration was measured by BCA kit (Perbio). 15-60 μg protein per sample was loaded onto a pre-cast NuPAGE Novex 4-12% Bis-Tris gel and separated using the Novex electrophoresis system (Invitrogen, Karsruhe, Germany). Subsequently, the proteins were transferred onto nitrocellulose membranes (Invitrogen) using the XCell II™ blot (Invitrogen). The immobilized proteins were visualized using MemCode reversible protein staining kit (Perbio). The membranes were blocked overnight at 4° C. in Roti-Block (Roth, Karlsruhe, Germany). For the detection of membrane-bound Aβ, primary detection antibodies 6E10 monoclonal antibodies (Biodesign, distributed by Dunn Labortechnik, Asbach, Germany) were used at a dilution of 1:1,000 for 1 h at ambient temperature on a roller shaker. The membranes were washed four times with PBS containing 0.05% Tween 20, and incubated for 1 h with horseradish peroxidase-conjugated secondary goat anti-mouse IgG (Perbio) at a dilution of 1:250,000. The blots were washed four times for 10 min, incubated for 5 min in SuperSignal West Dura Extended Duration Substrate working solution (Perbio) and exposed to an autoradiographic film (T-Mat Plus DG Film by Kodak). Alternatively, membranes were hybridized with rat anti-mouse F4/80 antibodies (Serotec, Dusseldorf; Germany). Equal protein loading of all membranes was assessed by reprobing with monoclonal antibodies against GAPDH (Acris, Hiddenhausen, Germany).

APP processing analysis in N2a cells. APP₆₉₅-transfected N2a cells (Marambaud et al., 2005) were grown in 1:1 DMEM/Opti-MEM supplemented with 5% FBS, penicillin and streptomycin, and 0.2 mg/ml G418. Cells were treated at confluency for 24 h with the indicated concentrations of CNI-1493. Medium was then changed and treatments were continued for another 2 h to allow Aβ secretion. Twenty microliters of conditioned medium were electrophoresed on 16.5% Tris-Tricine gels and transferred onto 0.2 gm nitrocellulose membranes. Membranes were then microwaved for 5 min in PBS, blocked in 5% fat-free milk in TBS, and incubated with 6E10 (Signet, 1:1000 in Pierce SuperBlock) overnight at 4° C. Cells were washed with PBS and solubilized in ice-cold HEPES buffer (25 mM HEPES, pH 7.4, 150 mM NaCl, 1× Complete protease inhibitor cocktail, Roche) containing 1% SDS. Ten micrograms of extracts were analyzed by western blot with 6E10, R1 (anti-APP C-terminal domain, reference 21), and LN27 (anti-APP₁₋₂₀₀, Zymed).

### Results and Discussion

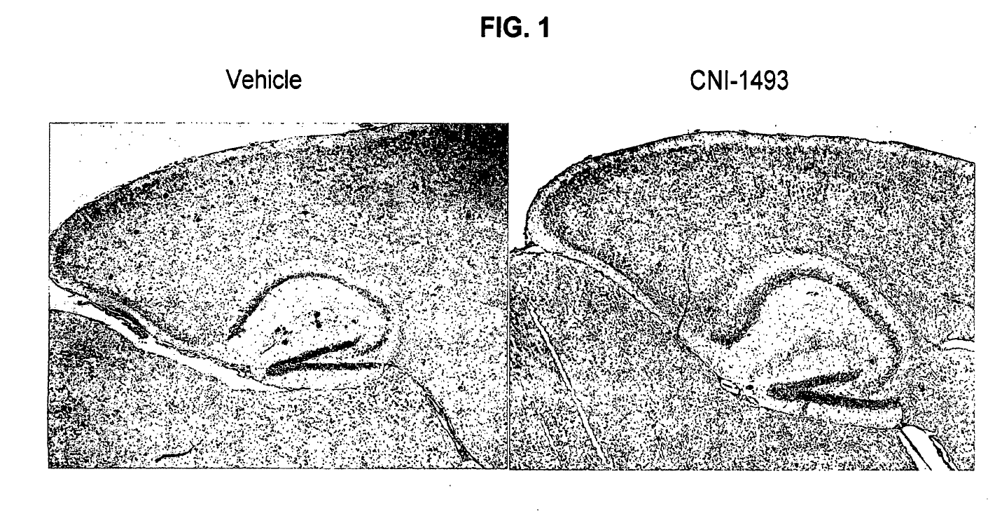

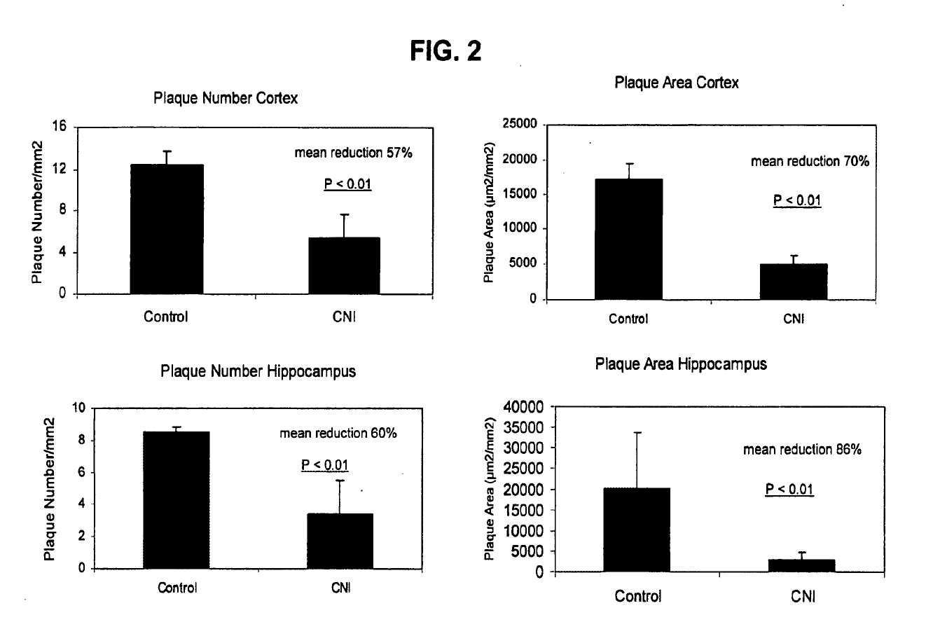

CNI-1493 prevents the formation of Aβ plaques in APP TgCRND8 transgenic mice. Treatment was initiated when the mice were almost 4 months old, the age at which plaque deposition typically begins in this model. Vehicle-treated mice developed significantly more plaques than CNI-1493 treated animals. (FIGS. 1 and 2). Evaluation of amyloid deposition demonstrated that CNI-1493 treatment resulted in a reduction of plaque number (plaque number divided by the area of interest in square mm) within the cortex by 57% and within the hippocampus by 60% compared with control animals. This effect by CNI-1493 was even more pronounced when we calculated the reduction of plaque area (area of the plaque in square micrometer divided by the area of interest in square mm). Here we obtained a reduction of 70% within cortex and 86% reduction within the hippocampus compared with control animals. We would like to emphasize that the magnitude of the decrease in Aβ-plaques by CNI-1493 was not only higher than what has recently been reported for the NSAID ibuprofen but was reached in only 2 months of treatment compared to ibuprofen treatment of 4 or 6 months (Lim et al., 2000; Yan et al., 2003).

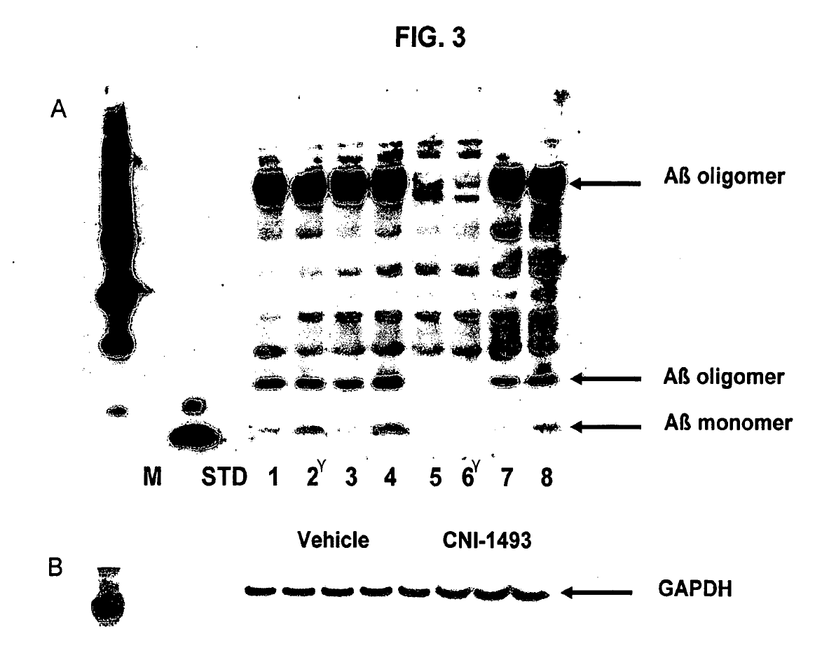

CNI-1493 effect on soluble Aβ content in APP TgCRND8 transgenic mice. After demonstrating a potent effect of CNI-1493 on plaque deposition in a relative short treatment period of 2 months, we next analyzed the brain cytosol fractions for a possible CNI-1493 dependent regulation of soluble Aβ. We obtained almost a complete reduction of the soluble Aβ isoforms in 2 out of 4 CNI-1493 treated animals by Western blot, whereas the remaining 2 animals showed no obvious alteration in soluble Aβ content compared with vehicle-treated animals (FIG. 3). Of note, all 4 animals which had received CNI-1493 showed significantly reduced Aβ plaque levels. However, the animals in which we detected almost a complete loss of soluble Aβ isoforms were also those with the highest rate of plaque reduction. The mechanisms by which anti-inflammatory agents, such as CNI-1493, affect AD risk in humans and amyloid pathology in animal models of the disease are likely to be complex and diverse. The unchanged levels of soluble Aβ isoform in 2 CNI-1493 treated animals suggests a dynamic process of CNI-1493 dependent plaque-deposition-interference which appears to be at least partly independent of the soluble Aβ levels.

CNI-1493 deactivates microglia cells in APP TgCRND8 transgenic mice. It has been debated that the principal cellular target of NSAIDs are microglia that are phenotypically activated as a consequence of amyloid deposition. CNI-1493 is a tetravalent guanylhydrazone that inhibits phosphorylation of p38 MAPK, c-Raf, and suppresses proinflammatory cytokine release from monocytes and macrophages (Cohen et al., 1997; Lowenberg et al., 2005; Bianchi et al., 1996; Wang et al., 1988; Tracy, 1998). Systemic administration of CNI-1493 is effective in the treatment of experimental autoimmune encephalomyelitis, Crohn's disease, cerebral ischemia, and arthritis (Martiney et al., 1998; Meistrell et al., 1997; Lowenberg et al., 2005; Akerlund et al., 1999).

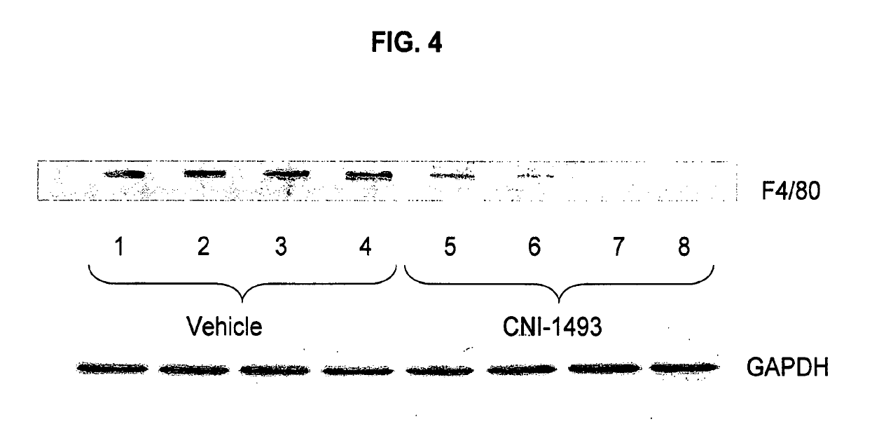

We evaluated microglia activation by analyzing the expression of the macrophage surface marker F4/80, which is elevated after activation of these cells (Ezekowitz et al., 1981). We obtained a decrease of F4/80 signal within the brain cytosol of all CNI-1493 treated animals in comparison with control animals (FIG. 4).

The CNI-1493 dependent decrease in total Aβ in N2a cells is unrelated to secretease activities. A number of NSAIDs have recently been reported to selectively regulate the processing of APP, and it has been argued that this effect may underlie their beneficial effects in AD (Weggen et al., 2001). NSAID treatment of APP overexpressing cells was reported to result in a preferential reduction in the production of Aβ42 and a parallel increase in Aβ38, although it had no effect on Aα40 (Id.).

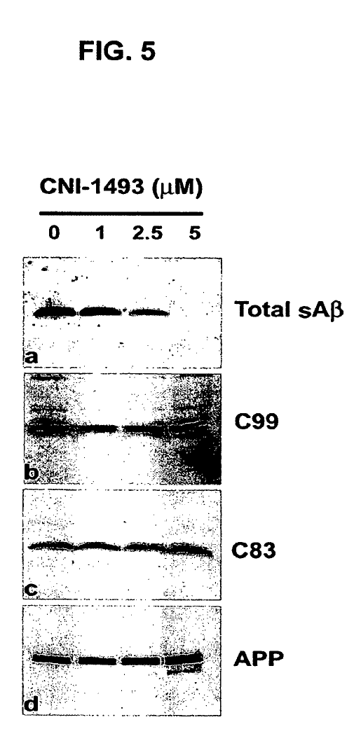

In this initial study, we tested whether CNI-1493 altered total Aβ production in N2a cells overexpressing human APP. CNI-l493 treatment resulted in a dose-dependent, dramatic reduction in the levels of total Aβ secreted into the medium (FIG. 5a). This result clearly implies that the CNI-1493 effect on APP processing is not restricted to reduction of Aβ42 but also includes the reduction of Aβ40. The observed decrease of total Aβ was not accompanied by reduced levels of APP production (FIG. 5d). Furthermore, CNI-1493 had no effect on the β- or γ-secretase cleavage of APP (FIG. 5b,c), which has recently been proposed for the mode of action of Aβ reduction by ibuprofen (Yan et al., 2003).

The major consideration raised by this study is the efficacy of the potent macrophage deactivator CNI-1493 in a murine Aβ plaque deposition model. We provide strong evidence that the principal effect of CNI-1493 treatment in APP transgenic TgCRND8 mice is a very significant reduction in plaque deposition after only 2 months of treatment. As anticipated, CNI-1493 treatment is accompanied by microglial deactivation. Our in vitro analysis of CNI-1493 treatment on APP processing in an APP overexpressing cell line indicates a profound dose-dependent decrease of total Aβ secretion. This effect appears to be completely unrelated from both the production of APP and changed β- or γ-secretase activities.

### EXAMPLE 2.

### The Interaction of CNI-1492 with Synuclein

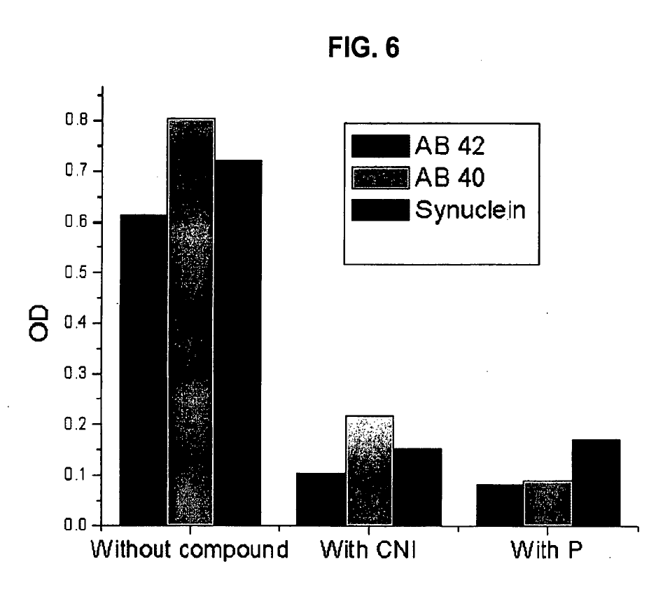

The ability of CNI-1493 to prevent aggregation of synuclein was tested by determining the recognition of synuclein by an anti-oligomer antibody (gift of Dr. C. Glabe, University of California at Irvine) in an ELISA assay. Combining either CNI-1492 or pentamidine (positive control) with either Aβ42, Aβ40 or synuclein reduced recognition by the antibody (FIG. 6), indicating that CNI-1492 prevents aggregation of those proteins.

### EXAMPLE 3

### The Interaction of CNI-1493 with Aβ42 in vitro and in Cells

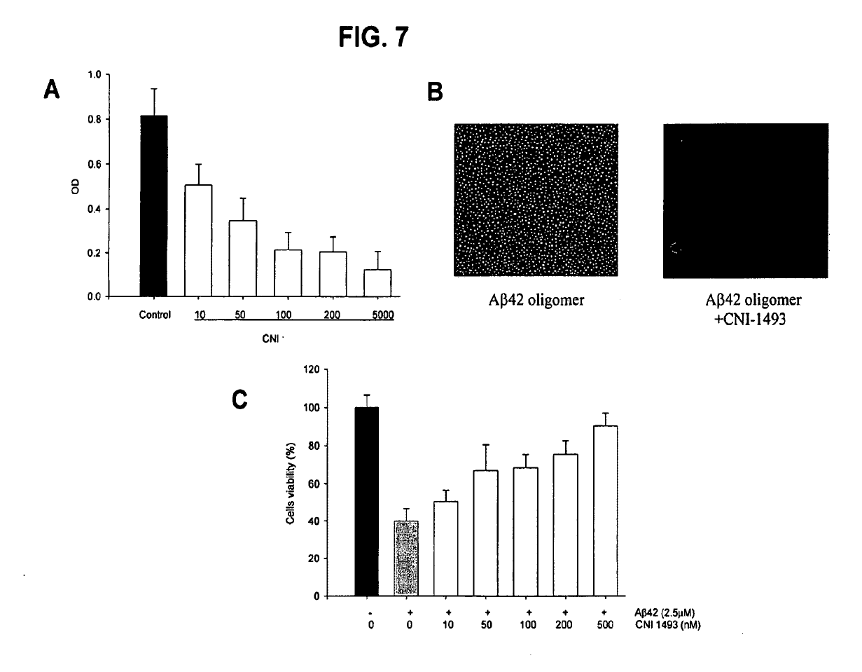

Aβ42 was combined with various concentrations of CNI-1493 and Aβ oligomers were quantified by ELISA using Aβ oligomer-specific antibodies. Increasing concentrations of CNI-1493 reduced the final OD in the ELISA (FIG. 7A), indicating that exposure to CNI-1493 disrupts Aβ oligomer assembly or the recognition of the oligomer by the anti-Aβ oligomer antibody. Electron microscope observation confirmed that, with CNI-1493 treatment (left panel, FIG. 7B), Aβ oligomers do not form the fibrillar aggregates that otherwise form in the absence of CNI-1493 (Right panel).

To determine whether the CNI-1493-treated form of Aβ is toxic, Aβ that had been pretreated with, or without, CNI-1493 was combined with SY5Y neuroblastoma cells. Toxicity was monitored using MTT (thiazolyl blue) reduction (mitochondrial succinate dehydrogenase activity) and lactate dehydrogenase activity (LDH). The MTT and LDH assays correlated with each other. As shown in FIG. 7C, soluble Aβ oligomers were toxic and addition of CNI-1493 reduced this toxicity in a dose-dependent matter. Additionally, the protective effect of CNI-1493 was examined in similar assays using primary neurons. As with the SY5Y cells, CNI-1493 protected the neurons from Aβ toxicity.

### REFERENCES

Aisen, P S. 1997. Inflammation and Alzheimer's disease: mechanisms and therapeutic strategies. Gerontology. 43:143-149.

Akerlund, K., H. Erlandsson-Harris, K. J. Tracey, H. Wang, T. Fehniger, L. Klareskog, J. Andersson, and U. Andersson. 1999. Anti-inflammatory effects of a new TNF_inhibitor (CNI-1493) in collagen-induced arthritis in rats. J. Clin. Exp. Immunol. 115:32-41.

Akiyama, H., S. Barger, S. Barnum, B. Bradt, J. Bauer, G. M. Cole, N. R. Cooper, P. Eikelenboom, M. Emmerling, B. L. Fiebich, C. E. Finch, S. Frautschy, W. S. Griffin, H Hampel, M. Hull, G. Landreth, L. Lue, R. Mrak, I. R. Mackenzie, P. L. McGeer, M. K. O'Banion, J. Pachter, G. Pasinetti, C. Plata-Salaman, J. Rogers, R. Rydel, Y. Shen, W. Streit, R. Strohmeyer, I. Tooyoma, F. L. Van Muiswinkel, R. Veerhuis, D. Walker, S. Webster, B. Wegrzyniak, G. Wenk, T., and T. Wyss-Coray. 2000. Inflammation and Alzheimer's disease. Neurobiol.Aging. 21:383-421.

Bianchi, M., 0. Bloom, B. Sherry, J. Chesney, P. Cohen, P. Ulrich, T. Raabe, M. Bukrinsky, A. Cerami, and K. J. Tracey. 1996. Suppression of proinflammatory cytokines in monocytes by a tetravalent guanylhydrazone. J. Exp. Med. 83:927-936.

Chishti, M. A., D. S. Yang, C. Janus, A. L. Phinney, P. Home, J. Pearson, R. Strome, N. Zuker, J. Loukides, J. French, S. Turner, G. Lozza, M. Grilli, S. Kunicki, C. Morissette, J. Paquette, F. Gervais, C. Bergeron, P. E. Fraser, G. A. Carlson, P. S. George-Hyslop, and D. Westaway. 2001. Early-onset amyloid deposition and cognitive deficits in transgenic mice expressing a double mutant form of amyloid precursor protein 695. J. Biol. Chem. 276:21562-70.

Cohen, P. S., H. Schmidtmayerova, J. Dennis, L. Dubrovsky, B. Sherry, H. Wang, M. Bukrinsky, and K. J. Tracey. 1997. The critical role of p38 MAβ kinase in T cell HIV-1 replication. Mol. Med. 5:339-346.

Ezekowitz, R. A. B., J. Austyn, P. D. Stahl, and S. Gordon. 1981. Surface properties of bacillus Calmette-Guérin-activated mouse macrophages. Reduced expression of mannosespecific endocytosis, Fc receptors, and antigen F4/80 accompanies induction of Ia. J. Exp. Med 154:60.

Golde, T. E., C. B. Eckman, and S. G. Younkin. 2000. Biochemical detection of Aβ isoforms: implications for pathogenesis, diagnosis, and treatment of Alzheimer's disease. Biochim. Biophys. Acta. 1502:172-187.

Hardy, J. A., and G. A. Higgins. 1992. Alzheimer's disease: the amyloid cascade hypothesis. Science. 256:184-185.

in t'Veld, B. A., A. Ruitenberg, A. Hofman, L. J. Launer, C. M. van Duijn, T. Stijnen, M. M. Breteler, and B. H. Stricker. 2001. Nonsteroidal antiinflammatory drugs and the risk of Alzheimer's disease. N. Engl. J. Med. 345:1515-1521.

Lim, G. P., F. Yang, T. Chu, P. Chen, W. Beech, B. Teter, T. Tran, 0. Ubeda, K. Hsiao Ashe, S. A. Frautschy, and G. M. Cole. 2000. Ibuprofen suppresses plaque pathology and inflammation in a mouse model for Alzheimer's disease. J. Neurosci. 20:5709-5714.

Lowenberg, M., A. Verhaar, B. van den Blink, F. ten Kate, S. van Deventer, M. Peppelenbosch, and D. Hommes. 2005. Specific inhibition of c-Raf activity by semapimod induces clinical remission in severe Crohn's disease. J. Immunol. 175:2293-300.

Marambaud, P., H. Zhao H, and P. Davies. 2005. Resveratrol promotes clearance of Alzheimer's disease amyloid-beta peptides. J. Biol. Chem. 280:37377-82.

Martiney, J. A., A. J. Rajan, P. C. Charles, A. Cerami, P. C. Ulrich, S. MacPhail, K. J. Tracey, C. F. Brosnan. 1998. Prevention and treatment of experimental autoimmune encephalomyelitis by CNI-1493, a macrophage deactivating agent. J. Immunol. 160:5588-5595.

McGeer, P. L., Schulzer, M., and E. G. McGeer. 1996. Arthritis and anti-inflammatory agents as possible protective factors for Alzheimer's disease: a review of 17 epidemiologic studies. Neurology. 47:425-432.

Meistrell, M. E. III, G. I. Botchkina, H. Wang, E. DeSanto, K. M. Cockroft, O. Bloom, J. M. Vishnubhakat, P. Ghezzi, and K. J. Tracey. 1997. TNF is a brain-damaging cytokine in cerebral ischemia. Shock 8:341-348.

Selkoe, D. J. 2001. Alzheimer's disease: genes, proteins, and therapy. Physiol. Rev. 81:741-766.

Stewart, W. F., C. Kawas, M. Corrada, and E. J. Metter. 1997. Risk of Alzheimer's disease and duration of NSAID use. Neurology. 48:626-632.

Tracey, K. J. 1998. Suppression of TNF and other proinflammatory cytokines by the tetravalent guanylhydrazone CNI-1493. Prog. Clin. Biot. Res. 397:335-343.

Wang, H., M. Zhang, M. Bianchi, B. Sherry, A. Sarna, and K. J. Tracey. 1988. Fetuin (_-2-HS-Glycoprotein) opsonises cationic macrophage-deactivating molecules. Proc. Nail. Acad. Sci. USA. 95:14429-14434.

Weggen, S., J. L. Eriksen, P. Das, S. A. Sagi, R. Wang, C. U. Pietrzik, K. A. Findlay, T. E. Smith, M. P. Murphy, T. Bulter, D. E. Kang, N. Marquez-Sterling, T. E. Golde, and E. H. Koo. 2001. A subset of NSAIDs lower amyloidogenic Abeta42 independently of cyclooxygenase activity. Nature 414:212-216.

Wyss-Coray, T., F. Yan, A. H. Lin, J. D. Lambris, J. J. Alexander, R. J. Quigg, and E. Masliah. 2002. Prominent neurodegeneration and increased plaque formation in complement-inhibited Alzheimer's mice. Proc. Natl. Acad. Sci. U. S. A. 99:10837-10842.

Yan, Q., J. Zhang, H. Liu, S. Babu-Khan, R. Vassar, A. L. Biere, M. Citron, and G. Landreth. 2003. J. Neurosci. 23:7504-9.

Younkin, S. G. 1998. The role of A beta 42 in Alzheimer's disease. J. Physiol.(Paris). 92:289-292.

Zandi, P. P., J. C. Anthony, K. M. Hayden, K. Mehta, L. Mayer, J. C. Breitner, and Cache County Study Investigators. 2002. Reduced incidence of AD with NSAID but not H2 receptor antagonists: the Cache County Study. Neurology. 59:880-886.
U.S. Pat. No. 5,599,984.
U.S. Pat. No. 5,750,573.
U.S. Pat. No. 5,753,684.
U.S. Pat. No. 5,849,794.
U.S. Pat. No. 5,854,289.
U.S. Pat. No. 5,859,062.
U.S. Pat. No. 6,008,255.
U.S. Pat. No. 6,022,900.
U.S. Pat. No. 6,180,676 B1.
U.S. Pat. No. 6,248,787 B1.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1-61. (canceled)

62. A compound having the structure: 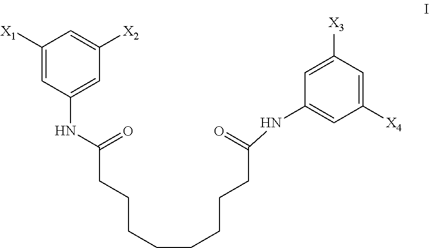
wherein
X₁, X₂, X₃ and X₄ are independently redGhyCH or redGhyCCH₃, where redGhyCH is NH₂(CNH)—NH—NH—CH₂— and redGhyCCH₃ is NH₂(CNH)—NH—NH—CH(CH₃)—.

63. The method of claim 1, wherein X₁, X₂, X₃ and X₄ is independently redGhyCH.

64. The method of claim 1, wherein X₁, X₂, X₃ and X₄ is independently redGhyCCH₃.

65. A composition comprising the compound of claim 1 and a pharmaceutically acceptable excipient.
